(19) United States
(12) Patent Application Publication
(10) Pub. No.: US 2022/0084656 A1
(43) Pub. Date: Mar. 17, 2022

(54) Systems and Methods for Automated Medical Image Identification for Protocols

(71) Applicant: FUJIFILM MEDICAL SYSTEMS U.S.A., INC., Morrisville, NC (US)

(72) Inventors: Richard Adam Hooker, Fishers, IN (US); Greg Hoofnagle, Cary, NC (US)

(21) Appl. No.: 17/024,460

(22) Filed: Sep. 17, 2020

(51) Int. Cl. G16H 30/20; G16H 50/70; G16H 70/20; G16H 50/30; G06T 7/00

(52) U.S. Cl. CPC G16H 30/20; G16H 50/70; G16H 70/20; A61B 5/7289; G06T 7/0012; G06T 2207/30168; G16H 50/30

(57) ABSTRACT

Method for identifying medical image records includes receiving a plurality of medical image records; receiving a protocol, the protocol including a plurality of measurements having an order; identifying a first medical image record of the plurality of medical image records, wherein a first measurement of the plurality of measurements can be performed on the first medical image record; displaying the first medical image record; receiving the first measurement; identifying a second medical image record of the plurality of medical image records, wherein a second measurement of the plurality of measurements can be performed on the second medical image record; displaying the second medical image record; and receiving the second measurement.

### BACKGROUND

### 1. Field of Disclosed Subject Matter

The disclosed subject matter is directed to systems and methods for automated medical image identification for protocols. For example, the systems and methods described herein can identify and present a user with the appropriate medical image to be used for each measurement in a protocol.

### 2. DESCRIPTION OF RELATED ART

In medical imaging, Picture Archiving and Communication Systems (“PACS”) are a combination of computers and networks dedicated to the storage, retrieval, presentation, and distribution of images. While medical information can be stored in a variety of formats, a common format of image storage is DICOM. DICOM is a standard in which, among other things, medical images and associated meta-data can be communicated from imaging modalities (e.g., ultrasound (“US”), x-ray (or x-rays' digital counterparts: computed radiography (“CR”) and digital radiography (“DR”)), computed tomography (“CT”), and magnetic resonance imaging (“MM”) apparatuses) to remote storage and/or client devices for viewing and/or other use.

During a medical exam, measurements or annotations can be performed on images corresponding to specific images, different phases or datasets. The plurality of measurements are referred to as a measurement protocol or protocol. Protocols often require medical professionals to view different medical images for one or more of the measurements. For example, left ventricle ejection fraction biplane method of disks (“MOD”) protocol can require measurements of the left ventricle volume in a four-chamber view of the heart and a two-chamber view of the heart during each of diastole and systole. Accordingly, the protocols can require professionals to switch between images to identify the best image for each measurement. Selecting the appropriate image can be time consuming and challenging because, for example, a professional must find the correct view and best heartbeat within that view (a set of images often includes multiple heart beats for a single view) to make the measurement.

Accordingly, there is a need for systems and methods for automated image identification for protocols.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended figures.

To achieve these and other advantages and in accordance with the purpose of the disclosed subject matter, as embodied and broadly described, the disclosed subject matter is directed to systems and methods for identifying medical image records. For example, a method for identifying medical image records includes receiving, at one or more computing devices, a plurality of medical image records; receiving, at the one or more computing devices, a protocol, the protocol including a plurality of measurements having an order; identifying, at the one or more computing devices, a first medical image record of the plurality of medical image records, wherein a first measurement of the plurality of measurements can be performed on the first medical image record; displaying, at the one or more computing devices, the first medical image record; receiving, at the one or more computing devices, the first measurement; identifying, at the one or more computing devices, a second medical image record of the plurality of medical image records, wherein a second measurement of the plurality of measurements can be performed on the second medical image record; displaying, at the one or more computing devices, the second medical image record; and receiving, at the one or more computing devices, the second measurement. Each medical image record of the plurality of medical image records can be a DICOM Service-Object Pair Instance.

Each measurement of the protocol can be associated with a view. Identifying the first medical image record can be based at least in part on the view associated with the first medical image record; and wherein identifying the second medical image record can be based at least in part on the view associated with the second medical image record. Each measurement of the protocol can be associated with a cardiac cycle point. Identifying the first medical image record can be based at least in part on the cardiac cycle point associated with the first measurement; and wherein identifying the second medical image record can be based at least in part on the cardiac cycle point associated with the second measurement. Identifying the first medical image record can be based at least in part on an image quality of the first medical image record; and wherein identifying the second medical image record can be based at least in part on an image quality of the second medical image record.

In accordance with the disclosed subject matter, one or more computer-readable non-transitory storage media embodying software are provided. The software can be operable when executed to: receive, at one or more computing devices, a plurality of medical image records; receive, at the one or more computing devices, a protocol, the protocol including a plurality of measurements having an order; identify, at the one or more computing devices, a first medical image record of the plurality of medical image records, wherein a first measurement of the plurality of measurements can be performed on the first medical image record; display, at the one or more computing devices, the first medical image record; receive, at the one or more computing devices, the first measurement; identify, at the one or more computing devices, a second medical image record of the plurality of medical image records, wherein a second measurement of the plurality of measurements can be performed on the second medical image record; display, at the one or more computing devices, the second medical image record; and receive, at the one or more computing devices, the second measurement.

In accordance with the disclosed subject matter, a system including one or more processors; and a memory coupled to the processors including instructions executable by the processors are provided. The processors can be operable when executing the instructions to: receive, at one or more computing devices, a plurality of medical image records; receive, at the one or more computing devices, a protocol, the protocol including a plurality of measurements having an order; identify, at the one or more computing devices, a first medical image record of the plurality of medical image records, wherein a first measurement of the plurality of measurements can be performed on the first medical image record; display, at the one or more computing devices, the first medical image record; receive, at the one or more computing devices, the first measurement; identify, at the one or more computing devices, a second medical image record of the plurality of medical image records, wherein a second measurement of the plurality of measurements can be performed on the second medical image record; display, at the one or more computing devices, the second medical image record; and receive, at the one or more computing devices, the second measurement.

### DRAWINGS

FIG. 1 shows a hierarchy of medical image records that can be used for identifying medical image records, in accordance with the disclosed subject matter.

FIG. 2 shows the architecture of a system for identifying medical image records, in accordance with the disclosed subject matter.

FIG. 3 shows a graphical user interface (“GUI”) of a system for identifying medical image records, in accordance with the disclosed subject matter.

FIG. 4 shows the GUI of a system for identifying medical image records including a measurement performed thereon, in accordance with the disclosed subject matter.

FIGS. 5A and 5B show a GUI of a system for identifying medical image records, in accordance with the disclosed subject matter.

FIG. 6 is a flow chart of a method for identifying medical image records in accordance with the disclosed subject matter.

### DETAILED DESCRIPTION

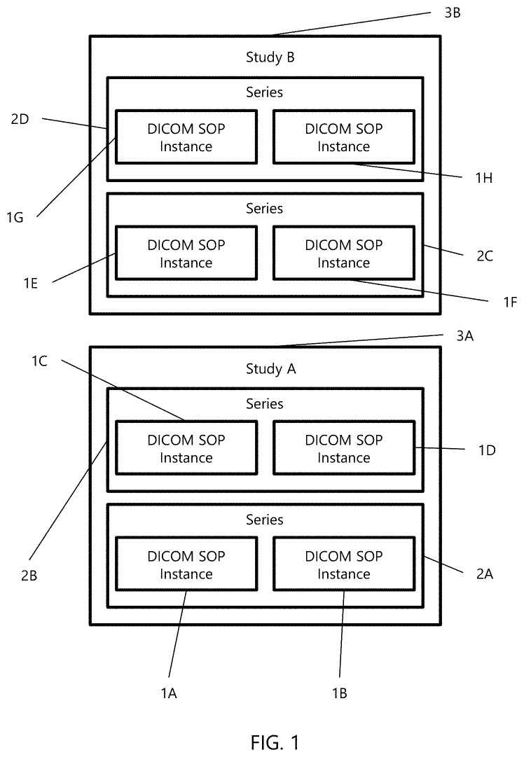

Reference will now be made in detail to various exemplary embodiments of the disclosed subject matter, exemplary embodiments of which are illustrated in the accompanying figures. For purpose of illustration and not limitation, the systems and methods are described herein for automated medical image identification for protocols. As used in the description and the appended claims, the singular forms, such as “a,” “an,” “the,” and singular nouns, are intended to include the plural forms as well, unless the context clearly indicates otherwise. Accordingly, as used herein, the term medical image record can refer to one medical image record, or a plurality of medical image records. For example, and with reference to FIG. 1 for purpose of illustration and not limitation, as referred to herein a medical image record can include a single DICOM SOP Instance (also referred to as “DICOM Instance” and “DICOM image”) 1 (e.g., 1A-1H), one or more DICOM SOP Instances 1 (e.g., 1A-1H) in one or more Series 2 (e.g., 2A-D), one or more Series 2 (e.g., 2A-D) in one or more Studies 3 (e.g., 3A, 3B), and one or more Studies 3 (e.g., 3A, 3B). The methods and systems described herein can be used with medical image records stored on PACS, however, a variety of records are suitable for the present disclosure and records can be stored in any system, for example a Vendor Neutral Archive (“VNA”). The disclosed systems and methods can be performed in an automated fashion (i.e., no user input once the method is initiated) or in a semi-automated fashion (i.e., with some user input once the method is initiated).

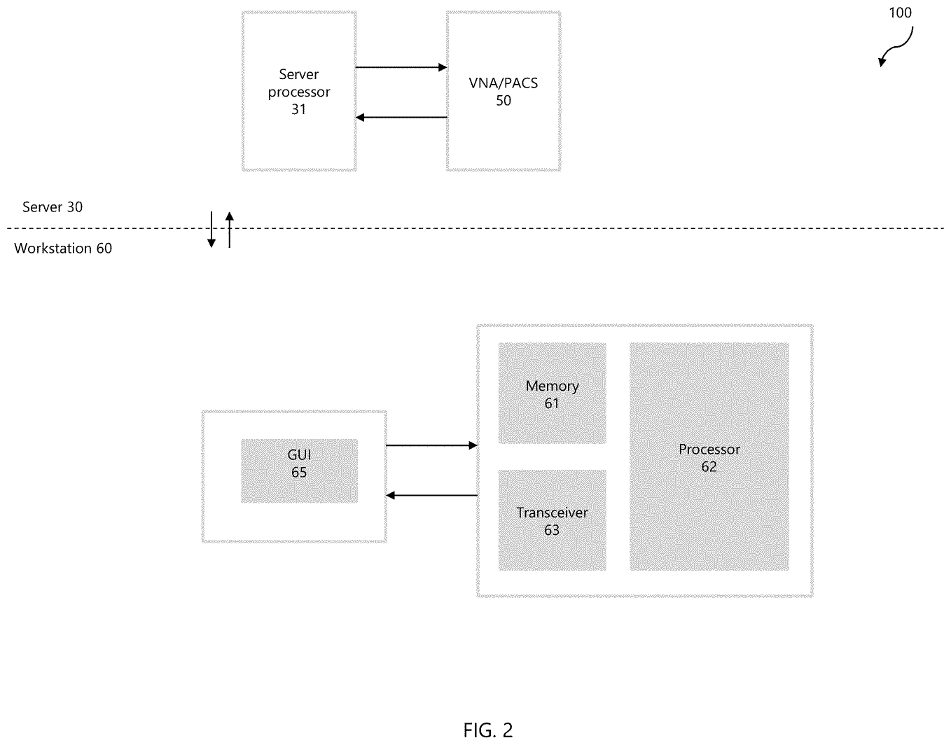

Referring to FIGS. 2-4 for purpose of illustration and not limitation, the disclosed system 100 can be configured to identify a first medical image record 10 (e.g., 10A-10D) for use in a first measurement 14 (e.g., 14A-14D) for a measurement protocol (also referred to herein as “protocol”) 13 (e.g., 13A-13B) and display the first medical image record 10 (e.g., 10A-10D) to the user to make the measurement 14 (e.g., 14A-14D) thereon. The systems 100 can then identify a second medical image record 10 (e.g., 10A-10D) for use in a second measurement 14 (e.g., 14A-14D) for a measurement protocol 13 (e.g., 13A-13B) and display the second medical image record 10 (e.g., 10A-10D) to the user to make the measurement 14 (e.g., 14A-14D) thereon. The system 100 can proceed accordingly for each measurement 14 (e.g., 14A-14D) in the measurement protocol 13 (e.g., 13A-13B). The system 100 can include one or more computing devices defining a server 30 and user workstation 60. The user workstation 60 can be coupled to the server 30 by a network. The network, for example, can be a Local Area Network (“LAN”), a Wireless LAN (“WLAN”), a virtual private network (“VPN”), or any other network that allows for any radio frequency or wireless type connection. For example, other radio frequency or wireless connections can include, but are not limited to, one or more network access technologies, such as Global System for Mobile communication (“GSM”), Universal Mobile Telecommunications System (“UMTS”), General Packet Radio Services (“GPRS”), Enhanced Data GSM Environment (“EDGE”), Third Generation Partnership Project (“3GPP”) Technology, including Long Term Evolution (“LTE”), LTE-Advanced, 3G technology, Internet of Things (“IOT”), fifth generation (“5G”), or new radio (“NR”) technology. Other examples can include Wideband Code Division Multiple Access (“WCDMA”), Bluetooth, IEEE 802.11b/g/n, or any other 802.11 protocol, or any other wired or wireless connection.

Workstation 60 can take the form of any known client device. For example, workstation 60 can be a computer, such as a laptop or desktop computer, a personal data or digital assistant (“PDA”), or any other user equipment or tablet, such as a mobile device or mobile portable media player. Server 30 can be a service point which provides processing, database, and communication facilities. For example, the server 30 can include dedicated rack-mounted servers, desktop computers, laptop computers, set top boxes, integrated devices combining various features, such as two or more features of the foregoing devices, or the like. Server 30 can vary widely in configuration or capabilities, but can include one or more processors, memory, and/or transceivers. Server 30 can also include one or more mass storage devices, one or more power supplies, one or more wired or wireless network interfaces, one or more input/output interfaces, and/or one or more operating systems. Server 30 can include additional data storage such as VNA/PACS 50, remote PACS, VNA, or other vendor PACS/VNA.

A user can be any person authorized to access workstation 60 and/or server 30, including a health professional, medical technician, researcher, or patient. In some embodiments a user authorized to use the workstation 60 and/or communicate with the server 30 can have a username and/or password that can be used to login or access workstation 60 and/or server 30.

Workstation 60 can include GUI 65, memory 61, processor 62, and transceiver 63. Medical image records 10 (e.g., 10A-10D) received by workstation 60 can be processed using one or more processors 62. Processor 62 can be any hardware or software used to execute computer program instructions. These computer program instructions can be provided to a processor of a general purpose computer to alter its function to a special purpose, a special purpose computer, application-specific integrated circuit (“ASIC”), or other programmable digital data processing apparatus, such that the instructions, which execute via the processor of the workstation 60 or other programmable data processing apparatus, implement the functions/acts specified in the block diagrams or operational block or blocks, thereby transforming their functionality in accordance with embodiments herein. The processor 62 can be a portable embedded micro-controller or micro-computer. For example, processor 62 can be embodied by any computational or data processing device, such as a central processing unit (“CPU”), digital signal processor (“DSP”), ASIC, programmable logic devices (“PLDs”), field programmable gate arrays (“FPGAs”), digitally enhanced circuits, or comparable device or a combination thereof. The processor 62 can be implemented as a single controller, or a plurality of controllers or processors.

Workstation 60 can send and receive medical image records 10 (e.g., 10A-10B) from server 30 using transceiver 63. Transceiver 63 can, independently, be a transmitter, a receiver, or both a transmitter and a receiver, or a unit or device that can be configured both for transmission and reception. In other words, transceiver 63 can include any hardware or software that allows workstation 60 to communicate with server 30. Transceiver 63 can be either a wired or a wireless transceiver. When wireless, the transceiver 63 can be implemented as a remote radio head which is not located in the device itself, but in a mast. While FIG. 2 only illustrates a single transceiver 63, workstation 60 can include one or more transceivers 63. Memory 61 can be a non-volatile storage medium or any other suitable storage device, such as a non-transitory computer-readable medium or storage medium. For example, memory 61 can be a random-access memory (“RAM”), read-only memory (“ROM”), hard disk drive (“HDD”), erasable programmable read-only memory (“EPROM”), electrically erasable programmable read-only memory (“EEPROM”), flash memory or other solid-state memory technology. Memory 61 can also be a compact disc read-only optical memory (“CD-ROM”), digital versatile disc (“DVD”), any other optical storage, magnetic cassettes, magnetic tape, magnetic disk storage or other magnetic storage devices, or any other physical or material medium which can be used to tangibly store the desired information or data or instructions and which can be accessed by a computer or processor. Memory 61 can be either removable or non-removable.

Server 30 can include a server processor 31 and VNA/PACS 50. The server processor 31 can be any hardware or software used to execute computer program instructions. These computer program instructions can be provided to a processor of a general purpose computer to alter its function to a special purpose, a special purpose computer, ASIC, or other programmable digital data processing apparatus, such that the instructions, which execute via the processor of the client station or other programmable data processing apparatus, implement the functions/acts specified in the block diagrams or operational block or blocks, thereby transforming their functionality in accordance with embodiments herein. In accordance with the disclosed subject matter, the server processor 31 can be a portable embedded micro-controller or micro-computer. For example, server processor 31 can be embodied by any computational or data processing device, such as a CPU, DSP, ASIC, PLDs, FPGAs, digitally enhanced circuits, or comparable device or a combination thereof. The server processor 31 can be implemented as a single controller, or a plurality of controllers or processors.

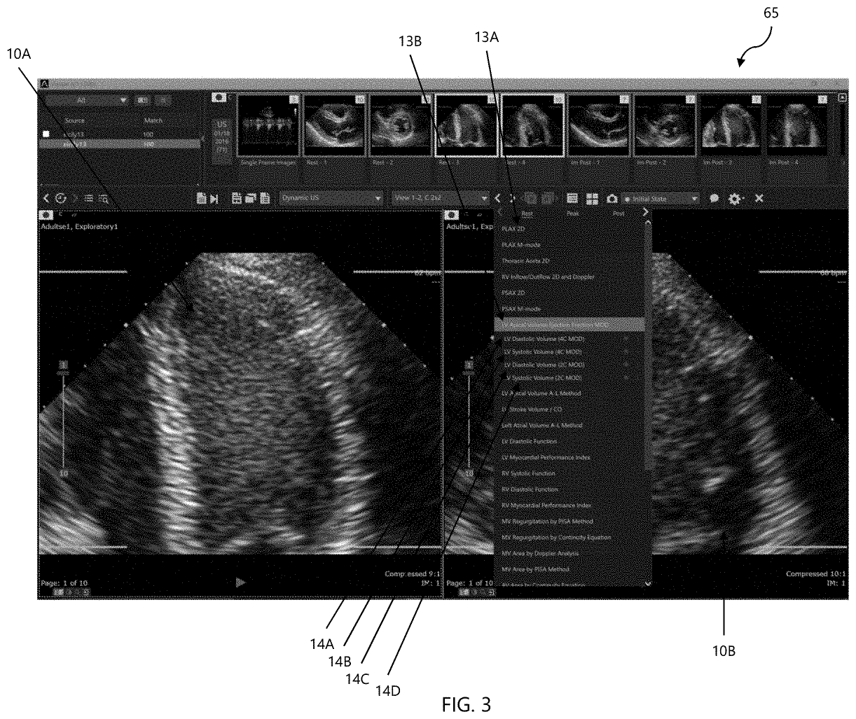

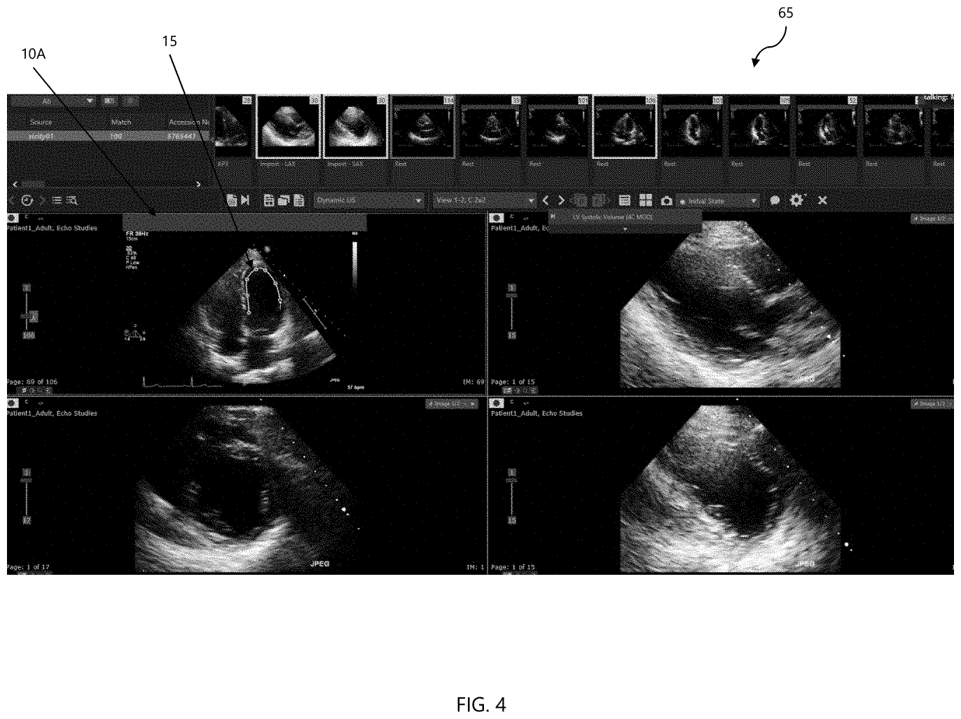

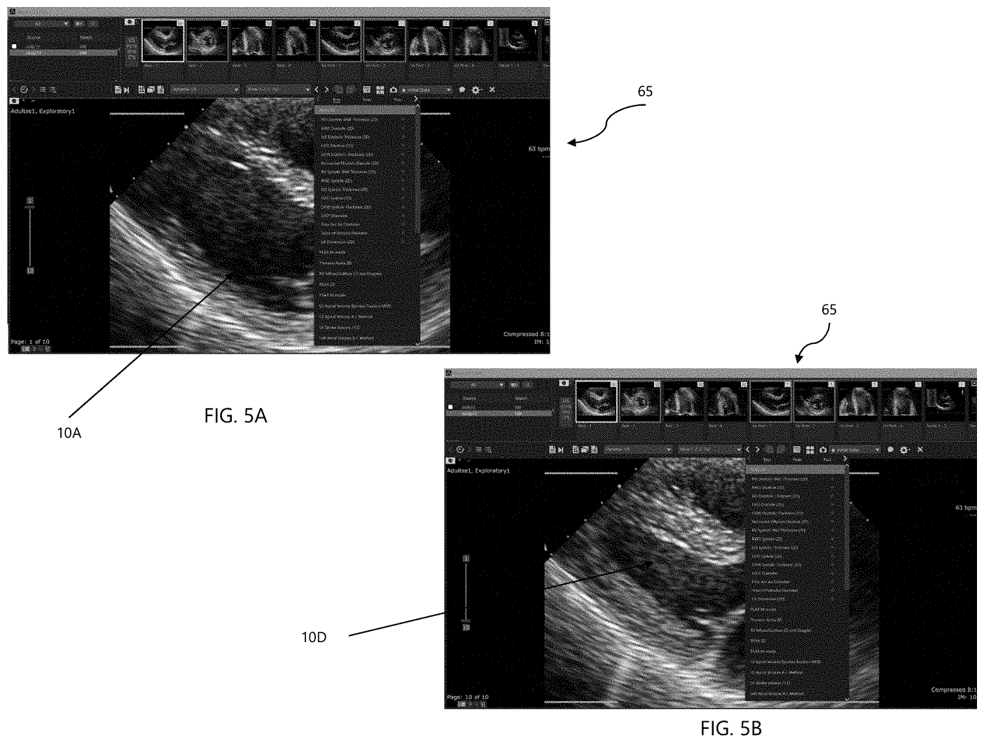

In operation, system 100 can be configured for automated medical image identification for protocols. FIGS. 3-5B show, for purpose of illustration and not limitation, a GUI 65 for the system 100 after having received a plurality of medical image records 10 (e.g., 10A-10D). In FIG. 3, GUI 65 is illustrated showing two medical image records 10A-10B, which are echocardiograms. Medical image record 10A shows a two chamber view and medical image record 10B shows a four chamber view, both during a rest phase. However, GUI 65 can provide any number of medical image records 10 (e.g., 10A-10D) and a variety of types of medical image records 10 (e.g., 10A-10D). For example, in FIG. 4 GUI 65 shows four medical image records 10 (e.g., 10A-10D) and in each of FIGS. 5A-5B, GUI 65 shows one medical image record 10 (e.g., 10A-10D).

The system 100 can receive a protocol 13 (e.g., 13A, 13B) when a user selects a protocol 13 (e.g., 13A, 13B), for example, from drop-down menu 12 (which can also be referred to as the Protocol Panel). For example, a user can select protocol 13B for Left Ventricle (“LV”) Apical Volume Ejection Fraction MOD which can include four measurements 14 (e.g., 14A-14D): 1) measurement 14A—LV Diastolic Volume (four chamber (“4C”) MOD); 2) measurement 14B—LV Systolic Volume (4C MOD); 3) measurement 14C—LV Diastolic Volume (two chamber (“2C”) MOD); and 4) measurement 14D—LV Systolic Volume (2C MOD). Each measurement 14 (e.g., 14A-14D) can be associated with a view (e.g., 4C and 2C views). Additionally, or alternatively, each measurement 14 (E.g., 14A-14D can be associated with a phase, such as a rest phase, an exercise phase, or a post exercise phase. Additionally or alternatively, each measurement 14 (e.g., 14A-14D) can associated with a cardiac cycle point (e.g., diastole and systole). For example, FIGS. 5A and 5B show medical image records 10C and 10D showing parasternal long-axis (“PLAX”) view during diastole and systole, respectively. One or more measurements 14 (e.g., 14A-D) within a protocol 13 (e.g., 13A, 13B) can be associated with the same or different view and/or cardiac cycle point. The measurements 14 (e.g., 14A-14D) can have an order, for example, measurement 14A can be first, measurement 14B can be second, measurements 14C can be third, and measurement 14D can be fourth. That is, the measurements 14A-14D can typically be performed in the order from first to fourth.

In accordance with the disclosed subject matter, system 100 can identify a medical image record 10 (e.g., 10A-10D) that can be used to perform the first measurement 14A. That is, first measurement 14A can be performed on the identified medical image 10A. Medical image 10A can include the correct view (e.g., 4C) and the correct cardiac cycle (e.g., diastole). The system 100 can identify the medical image record 10A based at least in part on the view associated with the measurement 14A and the cardiac cycle associated with the first measurement 14A. The system 100 can identify the medical image record 10A based at least in part on the image quality of the medical image 10A. The system 100 can identify the best medical image record 10A from a plurality of medical image records 10 (e.g., 10A-10D) that include the correct view, the correct cardiac cycle, and best image quality for performing the measurement 14A. Identifying the correct view and/or phase displayed in a medical image record 10 (e.g., 10A-10D) can be based on specific DICOM header tags. Identifying the correct cardiac cycle point displayed in a medical image record 10 (e.g., 10A-10D) can be based on a software algorithm. System 100 can display medical image record 10A on GUI 65. The medical image record 10A can be displayed with other medical image records 10 (e.g., 10B-10C) or can be displayed as the only visible medical image record 10A. Automatically identifying and displaying the best medical image record 10A can save a user time because the user will not need to search for the correct medical image record 10 (e.g., 10A-10D) or select a medical image record 10 (e.g., 10A-10D) from a set of eligible medical image records 10 (e.g., 10A-10D).

The user can perform measurement 14A on the medical image record 10A using a point-and-click system 15 that can trace the required measurement. System 100 can receive the measurement 14A performed by the user via GUI 65. System 100 can identify a medical image record 10 (e.g., 10A-10D) that can be used to perform the second measurement 14B. The medical image a record 10 (e.g., 10A-10D) can be the same medical image record 10A or a different medical image record 10 (e.g., 10B-10D) used for the first measurement 14A. The medical image record 10 (e.g., 10A-10D) can be displayed on GUI 65. For example, if the second measurement 14B requires the same medical image record 10A, the medical image record 10A can remain on GUI 65. If the second measurement 14B requires a different medical image record 10 (e.g., 10B-10C), system 100 can automatically switch to the correct medical image record 10 (e.g., 10B-10C). The measurement 14B can be performed by the user and received by the system as set forth above with regard to measurement 14A. System 100 can repeat the process for each measurement 14 (e.g., 14A-14D) within protocol 13B in order.

In accordance with the disclosed subject matter, measurements or annotations can be performed on medical image records 10 (e.g., 10A-10D) corresponding to different phases or datasets. For example, a phase (or stage) can refer to medical image records 10 (e.g., 10A-10D) taken at rest, during (or immediately following) exercise, and after a rest period following exercise. The phase of the study during which the medical image records 10 (e.g., 10A-10D) was captured from can be stored in the DICOM header for a PACS system to correctly arrange in playback order. The stage data can be used to also dynamically create a protocol panel for quantitative analysis. By providing a flexible solution, system 100 would not need to be configured each time an imaging protocol is updated to add/edit stages or research protocols are introduced.

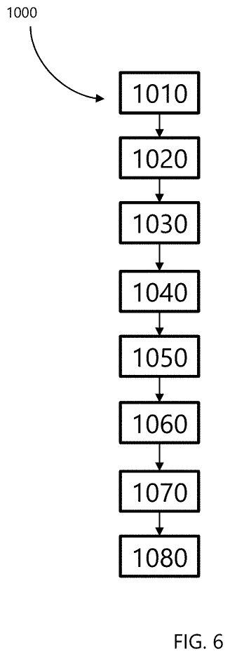

FIG. 6 illustrates an example method 1000 for identifying medical image records. The method 1000 can begin at step 1010, where the method includes receiving, at one or more computing devices, a plurality of medical image records. At step 1020 the method can include receiving, at the one or more computing devices, a protocol, the protocol including a plurality of measurements having an order. At step 1030 the method can include identifying, at the one or more computing devices, a first medical image record of the plurality of medical image records, wherein a first measurement of the plurality of measurements can be performed on the first medical image record. At step 1040 the method can include displaying, at the one or more computing devices, the first medical image record. At step 1050 the method can include receiving, at the one or more computing devices, the first measurement. At step 1060 the method can include identifying, at the one or more computing devices, a second medical image record of the plurality of medical image records, wherein a second measurement of the plurality of measurements can be performed on the second medical image record. At step 1070 the method can include displaying, at the one or more computing devices, the second medical image record. At step 1080 the method can include receiving, at the one or more computing devices, the second measurement. In accordance with the disclosed subject matter, the method can repeat one or more steps of the method of FIG. 6, where appropriate. Although this disclosure describes and illustrates particular steps of the method of FIG. 6 as occurring in a particular order, this disclosure contemplates any suitable steps of the method of FIG. 6 occurring in any suitable order. Moreover, although this disclosure describes and illustrates an example method for identifying medical image records including the particular steps of the method of FIG. 6, this disclosure contemplates any suitable method for identifying medical image records including any suitable steps, which can include all, some, or none of the steps of the method of FIG. 6, where appropriate. Furthermore, although this disclosure describes and illustrates particular components, devices, or systems carrying out particular steps of the method of FIG. 6, this disclosure contemplates any suitable combination of any suitable components, devices, or systems carrying out any suitable steps of the method of FIG. 6.

As described above in connection with certain embodiments, certain components, e.g., server 30 and workstation 60, can include a computer or computers, processor, network, mobile device, cluster, or other hardware to perform various functions. Moreover, certain elements of the disclosed subject matter can be embodied in computer readable code which can be stored on computer readable media and which when executed can cause a processor to perform certain functions described herein. In these embodiments, the computer and/or other hardware play a significant role in permitting the system and method for identifying medical image records. For example, the presence of the computers, processors, memory, storage, and networking hardware provides the ability to identify medical image records in a more efficient manner. Moreover, storing and saving the digital records cannot be accomplished with pen or paper, as such information is received over a network in electronic form.

The subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium also can be, or may be included in, one or more separate physical components or media (e.g., multiple CDs, disks, or other storage devices).

The term processor encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA or an ASIC. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA or an ASIC.

Processors suitable for the execution of a computer program can include, by way of example and not by way of limitation, both general and special purpose microprocessors. Devices suitable for storing computer program instructions and data can include all forms of non-volatile memory, media and memory devices, including by way of example but not by way of limitation, semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Additionally, as described above in connection with certain embodiments, certain components can communicate with certain other components, for example via a network, e.g., a local area network or the internet. To the extent not expressly stated above, the disclosed subject matter is intended to encompass both sides of each transaction, including transmitting and receiving. One of ordinary skill in the art will readily understand that with regard to the features described above, if one component transmits, sends, or otherwise makes available to another component, the other component will receive or acquire, whether expressly stated or not.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1) A method of identifying medical image records, comprising:
receiving, at one or more computing devices, a plurality of medical image records;
receiving, at the one or more computing devices, a protocol, the protocol including a plurality of measurements having an order;
identifying, at the one or more computing devices, a first medical image record of the plurality of medical image records, wherein a first measurement of the plurality of measurements can be performed on the first medical image record;
displaying, at the one or more computing devices, the first medical image record;
receiving, at the one or more computing devices, the first measurement;
identifying, at the one or more computing devices, a second medical image record of the plurality of medical image records, wherein a second measurement of the plurality of measurements can be performed on the second medical image record;
displaying, at the one or more computing devices, the second medical image record; and
receiving, at the one or more computing devices, the second measurement.

2) The method of claim 1, wherein each medical image record of the plurality of medical image records comprises a Digital Imaging and Communications in Medicine (“DICOM”) Service-Object Pair Instance.

3) The method of claim 1, wherein each measurement of the protocol is associated with a view.

4) The method of claim 3, wherein identifying the first medical image record is based at least in part on the view associated with the first medical image record; and wherein identifying the second medical image record is based at least in part on the view associated with the second medical image record.

5) The method of claim 1, wherein each measurement of the protocol is associated with a cardiac cycle point.

6) The method of claim 5, wherein identifying the first medical image record is based at least in part on the cardiac cycle point associated with the first measurement; and wherein identifying the second medical image record is based at least in part on the cardiac cycle point associated with the second measurement.

7) The method of claim 1, wherein identifying the first medical image record is based at least in part on an image quality of the first medical image record; and wherein identifying the second medical image record is based at least in part on an image quality of the second medical image record.

8) One or more computer readable non-transitory storage media embodying software that is operable when executed to:
receive, at one or more computing devices, a plurality of medical image records;
receive, at the one or more computing devices, a protocol, the protocol including a plurality of measurements having an order;
identify, at the one or more computing devices, a first medical image record of the plurality of medical image records, wherein a first measurement of the plurality of measurements can be performed on the first medical image record;
display, at the one or more computing devices, the first medical image record;
receive, at the one or more computing devices, the first measurement;
identify, at the one or more computing devices, a second medical image record of the plurality of medical image records, wherein a second measurement of the plurality of measurements can be performed on the second medical image record;
display, at the one or more computing devices, the second medical image record; and
receive, at the one or more computing devices, the second measurement.

9) The media of claim 8, wherein each medical image record of the plurality of medical image records comprises a Digital Imaging and Communications in Medicine (“DICOM”) Service-Object Pair Instance.

10) The media of claim 8, wherein each measurement of the protocol is associated with n view.

11) The media of claim 10, wherein the software is further operable to identify the first medical image record based at least in part on the view associated with the first medical image record; and wherein the software is further operable to identify the second digital image based at least in part on the view associated with the second digital image.

12) The media of claim 8, wherein each measurement of the protocol is associated with a cardiac cycle point.

13) The media of claim 12, wherein the software is further operable to identify the first digital image based at least in part on the cardiac cycle point associated with the first measurement; and wherein the software is further operable to identify the second digital image based at least in part on the cardiac cycle point associated with the second measurement.

14) The media of claim 8, wherein the software is further operable to identify the first digital image based at least in part on an image quality of the first digital image; and wherein the software is further operable to identify the second digital image based at least in part on an image quality of the second digital image.

15) A system comprising: one or more processors; and a memory coupled to the processors comprising instructions executable by the processors, the processors being operable when executing the instructions to:
receive, at one or more computing devices, a plurality of medical image records;
receive, at the one or more computing devices, a protocol, the protocol including a plurality of measurements having an order;
identify, at the one or more computing devices, a first digital image of the plurality of digital images, wherein a first measurement of the plurality of measurements can be performed on the first digital image;
display, at the one or more computing devices, the first digital image;
receive, at the one or more computing devices, the first measurement;
identify, at the one or more computing devices, a second digital image of the plurality of digital images, wherein a second measurement of the plurality of measurements can be performed on the second digital image;
display, at the one or more computing devices, the second digital image; and
receive, at the one or more computing devices, the second measurement.

16) The system of claim 15, wherein each medical image record of the plurality of medical image records comprises a Digital Imaging and Communications in Medicine (“DICOM”) Service-Object Pair Instance.

17) The system of claim 15, wherein each measurement of the protocol is associated with n view.

18) The system of claim 17, wherein the processors are further operable to identify the first digital image based at least in part on the view associated with the first digital image; and
wherein the processors are further operable to identify the second digital image based at least in part on the view associated with the second digital image.

19) The system of claim 15, wherein each measurement of the protocol is associated with a cardiac cycle point.

20) The system of claim 19, wherein the processors are further operable to identify the first digital image based at least in part on the cardiac cycle point associated with the first measurement; and wherein the processors are further operable to identify the second digital image based at least in part on the cardiac cycle point associated with the second measurement.

21) The system of claim 15, wherein the processors are further operable to identify the first digital image based at least in part on an image quality of the first digital image; and wherein the processors are further operable to identify the second digital image based at least in part on an image quality of the second digital image.
